(19) United States
(12) Patent Application Publication
(10) Pub. No.: US 2012/0238019 A1
(43) Pub. Date: Sep. 20, 2012

(54) STEM CELL CULTURE MEDIA FOR MAINTAINING STEMNESS OR INDUCING DIFFERENTIATION OF STEM CELLS

(75) Inventors: Dong-Youn HWANG, Seongnam-si (KR); Tae-Min YOON, Seoul (KR)

(73) Assignee: COLLEGE OF MEDICINE POCHON CHA UNIVERSITY INDUSTRY-ACADEMIC COOPERATION FOUNDATION, Pochon-siu (KR)

(21) Appl. No.: 13/051,660

(22) Filed: Mar. 18, 2011

(51) Int. Cl. C12N 5/0735; C12N 5/074

(52) U.S. Cl. 435/377; 435/375

(57) ABSTRACT

The present invention provides a medium for maintaining stemness of stem cells comprising a protein kinase C inhibitor in a basal medium; and a method for culturing stem cells while maintaining stemness thereof, using the same. And also, the present invention provides a medium for inducing differentiation of stem cells comprising a protein kinase C activator in a basal medium; and a method for inducing differentiation of stem cells, using the same.

### BACKGROUND OF THE INVENTION

1. Field of the Invention

The present invention relates to a medium for maintaining stemness of stem cells; and a method for culturing stem cells while maintaining stemness thereof, using the same. And also, the present invention relates to a medium for inducing differentiation of stem cells; and a method for inducing differentiation of stem cells, using the same.

2. Description of the Related Art

Stem cells refer to the cells which can divide for a long time without differentiation and also be differentiated into all cell types of body such as neurons, blood cells, and chondrocytes when needed.

Stem cells can be largely categorized into two types; embryonic stem cells derived from early embryos and adult stem cells existing in various tissues of adult body. In spite of certain functional differences, the two types of stem cells share common characteristics including capability to differentiate into more than one cell type.

Human embryonic stem cells (hESCs) retain two important characteristics: unlimited “self-renewal” and “pluripotency”, which refer to the abilities to continuously generate identical progeny cells throughout life and become any human cell type, respectively. These properties are the major components contributing to “sternness” of hESCs.

Till now, only a handful of signaling pathways are known to be implicated in maintaining stemness of hESCs; these include FGF (Fibroblast Growth Factor) and TGF-Beta (Transforming Growth Factor-Beta)/Activin signaling pathways. Therefore, stemness of human embryonic stem cells could be maintained by regulating activity of the components of these signaling pathways, such as SMAD 2/3 (Sma and MAD (Mothers Against Decapentaplegic) Related Protein-2/3), ALK4(Activin Receptor-Like Kinase-4), ALK5 (Activin Receptor-Like Kinase-5), ALK7 (Activin Receptor-Like Kinase-7), Nodal, Cripto, LEFTY1 (Left-right determination factor-1), and LEFTY2 (Left-right determination factor-2) (Xiao L, Yuan X, Sharkis S J., Stem Cells. 2006, 24(6), 1476-1486; Besser D., J Biol Chem. 2004, 279(43), 45076-45084; James D, Levine A J, Besser D, Hemmati-Brivanlou A., Development. 2005, 132(6), 1273-1282).

In addition, WNTs (Wingless-Type MMTV Integration Site Family Members) and WNT signaling pathway are implicated in maintaining stemness of hESCs. The regulation of this signaling pathway is mostly mediated by inhibition of GSK-3b (Glycogen Synthase Kinase-3b) activity and concomitant increase in beta-catenin level (Miyabayashi T, et. al, Proc Natl Acad Sci USA. 2007, 104(13), 5668-5773).

S1P (Sphingosine-1-Phosphate) was also reported to regulate hESC stemness either by initiating G-protein-mediated signaling cascade or by modulating PDGF(Platelet Derived-Growth Factor) signaling pathway (Chase L G, Firpo M T., Curr Opin Chem Biol. 2007, 11(4), 367-372; Inniss K, Moore H., Stem Cells Dev. 2006, 15(6), 789-796.).

Transcription factors such as Oct4 (Octamer Binding Transcription Factor-4), Nanog, Sox2 (SRY(Sex Determining Region-Y) Box-2) are also shown to play a critical role in maintaining stemness of hESCs.

Conventionally, hESCs are grown by co-culturing with mouse embryonic fibroblasts. Here, mouse embryonic fibroblasts (MEF) are used as feeder cells which provide growth factors or other proteins required for undifferentiated growth of hESCs. The natures of these factors are yet to be uncovered.

The hESCs grown by co-culturing with MEF are not eligible for most clinical applications due to the risk of xenopathogen contamination. Therefore, culturing hESCs in the absence of animal source may be one of the critical issues to be resolved before successful clinical applications of hESCs. In relation to this, several achievements have been made to develop feeder-free culture conditions in which the use of MEF is avoided in culturing hESCs. However, all the feeder-free culture conditions successfully developed so far contain animal-derived components such as bovine serum albumin and matrigel.

Therefore, one of the most urgent obstacles to overcome for clinical use of hESCs is establishing an efficient feeder-free hESC culture condition which does not contain animal-derived components. To develop such a feeder-free and xeno-free culture system, comprehensive understanding of signaling pathways regulating “hESC stemness” should be preceded.

### SUMMARY OF THE INVENTION

To uncover novel signaling cascades and ligands implicated in fate decision of hESCs, the present inventors performed various researches and came to the discovery that regulators (activators and inhibitors) of protein kinase C pathway play a critical role in either maintaining stemness or inducing differentiation of hESCs.

In accordance with an aspect of the present invention, there is provided a medium for maintaining stemness of stem cells comprising a protein kinase C inhibitor in a basal medium; and a method for culturing stem cells while maintaining stemness thereof, using the same.

In accordance with another aspect of the present invention, there is provided a medium for inducing differentiation of stem cells comprising a protein kinase C activator in a basal medium; and a method for inducing differentiation of stem cells, using the same.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other features and advantages of the present invention will become more apparent by describing in detail exemplary embodiments thereof with reference to the attached drawings in which:

FIG. 1 shows the results of evaluation of stemness of stem cells in a MEF-medium [DMEM-high glucose (WeIGENE, Korea)+10% (v/v) fetal bovine serum (FBS, WeIGENE, Korea)+1% (v/v) non essential amino acids (GIBCO)+0.1 mM beta-mercaptoethanol (GIBCO)+1% (v/v) Pennicillin/streptomycin (Caisson)] containing the PKC inhibitor (Gö6983).

FIG. 2 shows the results of evaluation of stemness of stem cells in a MEF non-conditioned medium [80% (v/v) DMEM/F12 (GIBCO), 20% (v/v) Knockout serum replacement (GIBCO), 1% (v/v) MEM non-essential amino acids (GIBCO), 0.1 mM β-mercaptoethanol (GIBCO), 1% (v/v) Pennicillin/streptomycin (Caisson)]] containing the PKC inhibitor (Gö6983).

FIG. 3 shows the results of evaluation of differentiation of stem cells in a mTeSR-1 (StemCell Technologies, Inc.) medium containing the PKC activator (12-O-tetradecanoylphorbol-13-acetate).

FIG. 4 shows the results of evaluation of differentiation of stem cells in a Matrigel (extracellular matrix) (BD Biosciences, USA)/MEF-conditioned medium (Home made; Serum-free media [80% (v/v) DMEM/F12 (GIBCO), 20% (v/v) Knockout serum replacement (GIBCO), 1% (v/v) MEM non-essential amino acids (GIBCO), 0.1 mM β-mercaptoethanol (GIBCO), 1% (v/v) Pennicillin/streptomycin (Caisson)] was conditioned by γ-irradiated CF-1 mouse embryonic fibroblasts (MEF) at 37° C. for 24 hours)and a CELLstart(extracellular matrix) (Invitrogen)/STEMPRO medium (Invitrogen) containing the PKC activator (12-O-tetradecanoylphorbol-13-acetate). FIG. 4 also shows the results of evaluation of differentiation of stem cells, using other stem cells (i.e., H9 hESC line).

### DETAILED DESCRIPTION OF THE INVENTION

The present invention will now be described more fully with reference to the accompanying drawings, in which exemplary embodiments of the invention are shown.

It is newly found that regulators (activators and inhibitors) of protein kinase C pathway play a critical role in either maintaining stemness or inducing differentiation of hESCs. The present inventors surprisingly found that a protein kinase C inhibitor involves in maintaining stemness of stem cells; and that a protein kinase C activator involves in inducing differentiation of stem cells

The present invention provides a medium for maintaining stemness of stem cells comprising a protein kinase C inhibitor in a basal medium.

The basal medium may be any conventional medium for culturing stem cells. The basal medium may be manually prepared according to conventional methods. And also, the basal medium may be a commercially available medium or a mixture thereof. For example, the basal medium may be selected from the group consisting of DMEM (Dulbecco's Modified Eagle's Medium; GIBCO), MEM (Minimal Essential Medium; GIBCO), BME (Basal Medium Eagle; GIBCO), RPMI 1640 (GIBCO), DMEM/F-12 (Dulbecco's Modified Eagle's Medium: Nutrient Mixture F-12; GIBCO), DMEM/F-10 (Dulbecco's Modified Eagle's Medium: Nutrient Mixture F-10; GIBCO), α-MEM (α-Minimal essential Medium; GIBCO), G-MEM (Glasgow's Minimal Essential Medium; GIBCO), IMDM (Isocove's Modified Dulbecco's Medium; GIBCO), and KnockOut DMEM (GIBCO). The basal medium may contain one or more supplements, which includes, but not limited to, KnockOut Serum Replacement (GIBCO), KnockOut SR XenoFree (GIBCO), KnockOut SR XenoFree Growth Factor Cocktail (GIBCO), N2 supplement (GIBCO), B27 supplement (GIBCO), and so on. Preferably, the basal medium may be a xenopathogen-free medium (i.e., xeno-free medium), in order to avoid any safety problem by materials derived from animal source. That is, it is preferable that the basal medium does not include xenopathgen(s), such as bovine serum albumin, and recombinant proteins purified from animal cells.

The protein kinase C inhibitor may be any agent(s) having an inhibitory activity against protein kinase C. The protein kinase C inhibitor includes, but not limited to, 2-[1-(3-dimethylaminopropyl)-5-methoxyindol-3-yl]-3-(1H-indol-3-yl)maleimide; 3-[1-[3-(dimethylamino)propyl]-5-methoxy-1H-indol-3-yl]-4-(1H-indol-3-yl)-1H-pyrrole-2,5-dione; 3-(1H-indol-3-yl)-4-[2-(4-methylpiperazin-1-yl)quinazolin-4-yl]pyrrole-2,5-dione); 3-{1-[3-(amidinothio)propyl]-1H-indol-3-yl}-3-(1-methyl-1H-indol-3-yl)maleimide methane sulfonate; 13-hydroxyoctadecadienoic acid; bisindolylmaleimide; 2-[1-(3-dimethylaminopropyl)indol-3-yl]-3-(indol-3-yl) maleimide (GF 109203X); 2,6-diamino-N-([1-(1-oxotridecyl)-2-piperidinyl]methyphexanamide; or 4′-demethylamino-4′-hydroxystaurosporine. Preferably, the protein kinase C inhibitor may be contained in an amount of 0.001 to 1000 uM, in the basal medium. When the protein kinase C inhibitor is contained in an amount of less than 0.001 uM, stemness-maintaining effect may be unsatisfactory. And also, when the protein kinase C inhibitor is contained in an amount of more than 1000 uM, cytotoxicity may occur.

In the medium for maintaining stemness of stem cells according to the present invention, the stem cells may be embryonic stem cells or adult stem cells, including neural stem cells, hepatic stem cells, hematopoietic stem cells, umbilical cord blood stem cells, epidermal stem cells, gastrointestinal stem cells, endothelial stem cells, muscle stem cells, mesenchymal stem cells, and pancreatic stem cells, but not limited thereto.

The present invention also includes, within its scope, a method for culturing stem cells while maintaining stemness thereof, using the medium mentioned in the above. The culturing stem cells may be performed in the presence of feeder cells (e.g., mouse embryonic fibroblasts (MEF) or in the absence of feeder cells. The present inventors also found that the use of the medium containing a protein kinase C inhibitor makes it possible to culture stem cells while maintaining stemness thereof, even in the absence of feeder cells. That is, the culturing stem cells may be performed under the feeder-free condition, so as to avoid any safety problem by feeder cells derived from animal source.

The present also provides a medium for inducing differentiation of stem cells comprising a protein kinase C activator in a basal medium.

The protein kinase C activator may be any agent(s) activating protein kinase C. The protein kinase C activator includes, but not limited to, 12-O-tetradecanoylphorbol-13-acetate; 1-hexylindolactam-V10; 6,11,12,14-tetrahydroxy-abieta-5,8,11,13-tetraene-7-one; 8-octyl-benzolactam-V9; acetyl-1-carnitine; phorbol 12-myristate 13-acetate; (R)-7-cyano-2,3,4,5-tetrahydro-1-(1H-imidazol-4-ylmethyl)-3-(phenylmethyl)-4-(2-thie nylsulfonyl)-1H-1,4-benzodiazepine (BMS-214662); Bryostatin 1; 1-oleoyl-2-acetyl-sn-glycerol; L-a-phosphatidylinositol-3,4-bisphosphate; 1-stearoyl-2-arachidonoyl-sn-glycerol; and resiniferonol 9,13,14-ortho-phenylacetate. Preferably, the protein kinase C activator may be contained in an amount of 0.001 to 1000 uM, in the basal medium. When the protein kinase C activator is contained in an amount of less than 0.001 uM, differentiation-inducing effect may be unsatisfactory. And also, when the protein kinase C activator is contained in an amount of more than 1000 uM, cytotoxicity may be occurred.

As a basal medium in the medium for inducing differentiation of stem cells, one or more basal media mentioned in the above may be used. For example, the basal medium may be selected from the group consisting of DMEM (Dulbecco's Modified Eagle's Medium; GIBCO), MEM (Minimal Essential Medium; GIBCO), BME (Basal Medium Eagle; GIBCO), RPMI 1640 (GIBCO), DMEM/F-12 (Dulbecco's Modified Eagle's Medium: Nutrient Mixture F-12; GIBCO), DMEM/F-10 (Dulbecco's Modified Eagle's Medium: Nutrient Mixture F-10; GIBCO), α-MEM (α-Minimal essential Medium; GIBCO), G-MEM (Glasgow's Minimal Essential Medium; GIBCO), IMDM (Isocove's Modified Dulbecco's Medium; GIBCO), and KnockOut DMEM (GIBCO). The basal medium may contain one or more supplements, which mentioned in the above. And also, the basal medium for inducing differentiation of stem cells is preferably a xenopathgen-free medium (i.e., xeno-free medium), in order to avoid any safety problem by materials derived from animal source. That is, it is preferable that the basal medium does not include xenopathgen(s), such as bovine serum albumin, and recombinant proteins purified from animal cells.

The present invention also includes, within its scope, a method for inducing differentiation of stem cells, using the medium mentioned in the above. The culturing stem cells may be performed in the presence of feeder cells (e.g., mouse embryonic fibroblasts (MEF) or in the absence of feeder cells. The present inventors also found that the use of the medium containing a protein kinase C activator makes it possible to culture stem cells for inducing differentiation thereof, even in the absence of feeder cells. That is, the culturing the stem cells may be performed under the feeder-free condition, so as to avoid any safety problem by feeder cells derived from animal source.

According to the present invention, a xeno-free and/or feeder-free medium may be prepared using a medium comprising a protein kinase C inhibitor or a protein kinase C activator. That is, the medium according to the present invention may be useful for culturing stem cells without differentiation thereof under xeno-free and/or feeder-free condition; or for differentiating stem cells without teratoma formation under xeno-free and/or feeder-free condition. Especially, when differentiation to a specific cell lineage for cell therapy is performed in a medium comprising a protein kinase C activator according to the present invention, the existence of undifferentiated stem cells in the resulting cells can be minimized, thereby avoiding teratoma formation.

The present invention will be described in further detail with reference to the following examples. These examples are for illustrative purposes only and are not intended to limit the scope of the present invention.

### EXAMPLE 1

### Evaluation of Stemness of Stem Cells in the Presence of PKC Inhibitor

(1) Culture in MEF-Medium Containing PKC Inhibitor

Colony clumps (5 colony clumps) of human embryonic stem cells (CHA6 cell line, CHA Medical University, Korea) were seeded to each wells of a 96-well plate coated with Matrigel (BD Biosciences, USA). A differentiation-inducing medium, i.e., MEF medium [Dulbecco's Modified Eagle Medium (DMEM) containing high glucose (WeIGENE, Korea) supplemented with 10% (v/v) FBS (WeIGENE, Korea), 1% (v/v) Penicillin/streptomycin (Caisson), 1% (v/v) NEAA (GIBCO), 0.1 mM beta-mercaptoethanol (GIBCO)], was added to the each wells, which were then cultured at about 37° C., in about 5.0% CO₂ incubator. The culture was performed for 5 to 7 days, while replacing the medium with a new medium containing 5 uM of 3-[1-[3-(dimethylamino)propyl]-5-methoxy-1H-indol-3-yl]-4-(1H-indol-3-yl)-1H-pyrrole-2,5-dione)(Gö6983, Calbiochem) as a PKC inhibitor, everyday.

As a control, human embryonic stem cells were cultured in the MEF medium without the PKC inhibitor. And also, human embryonic stem cells were cultured in a differentiation-inhibiting medium, i.e., mTeSR-1 medium containing bFGF, TGF-beta, LiCl, GABA, and pipecolic acid (StemCell Technologies, USA).

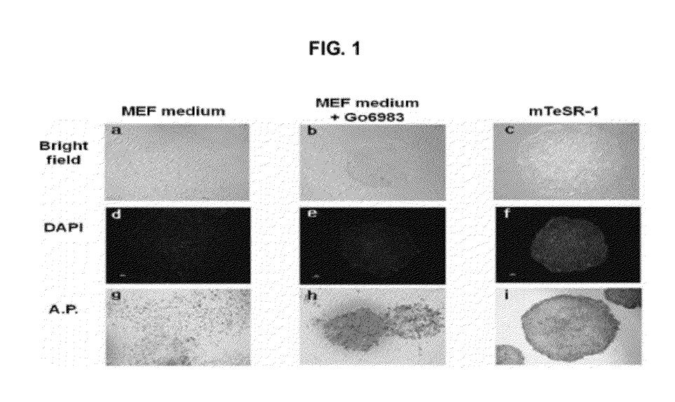

For evaluating differentiation of the stem cells, shape of the cells was observed with an optical microscope (a to c in FIG. 1) and existence and distribution of the cells were observed through nucleus-staining with DAPI (d to f in FIG. 1). The cells cultured in the MEF medium were differentiated and broadly distributed (a and d in FIG. 1). In contrast, the cells cultured in the MEF medium containing the PKC inhibitor maintained colony shapes, which were conglomerating each other (b and e in FIG. 1). The cells cultured in the differentiation-inhibiting medium (i.e., the mTeSR-1 medium) also conglomerated each other in colony shapes without differentiation (c and f in FIG. 1).

And also, for evaluating expression of alkaline phosphatase which is one of the un-differentiated cell markers, alkaline phosphatase staining was performed as followings: the cells were fixed with 4% para-formaldehyde and then washed with lx TBST (20 mM Tris-HCl, pH 7.4, 0.15M NaCl, 0.05% Tween-20). The cells were treated with a mixture of Fast red violet, naphtol, and H₂O (2:1:1, v/v/v) for 15 minutes, washed with 1× TBST, and then 1× PBS was added thereto. The results were shown in g to i in FIG. 1. The cells cultured in the MEF medium were differentiated and broadly distributed (g in FIG. 1). In contrast, the cells cultured in the MEF medium containing the PKC inhibitor maintained colony shapes and showed significant increase in alkaline phosphatase expression level (h in FIG. 1). These results indicate that the PKC inhibitor inhibits differentiation of stem cells and maintains stemness thereof. The cells cultured in the differentiation-inhibiting medium (i.e., the mTeSR-1 medium) also showed no differentiation and significant increase in alkaline phosphatase expression level (i in FIG. 1).

(2) Culture in Non-Conditioned Medium Containing PKC Inhibitor

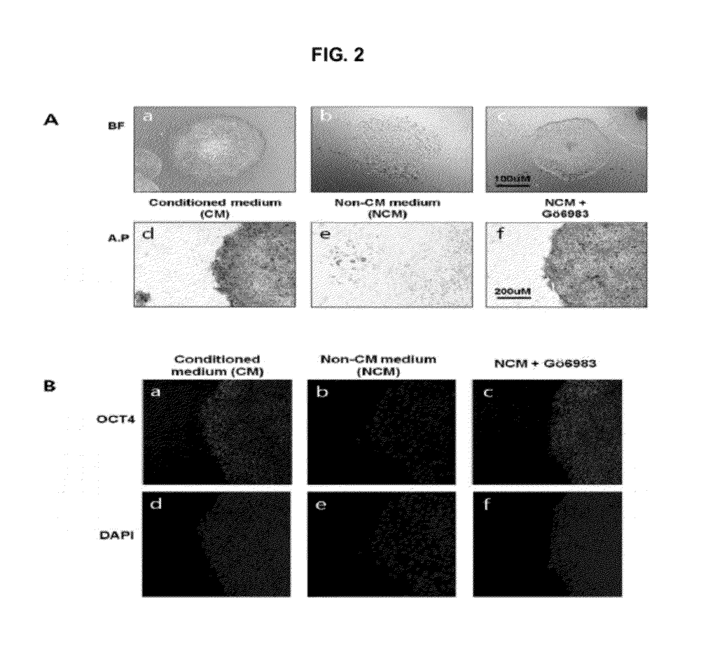

Evaluations of stemness were also performed in accordance with the same procedures as in the above (1), using a non-conditioned medium [DMEM/F-12 high-glucose (GIBCO) supplemented with 20% (v/v) knockout serum replacement (GIBCO), 1% (v/v) Penicillin/streptomycin (Caisson), 1% (v/v) NEAA(GIBCO), and 0.1 mM beta-mercaptoethanol (GIBCO)], instead of the MEF medium. In addition, expression levels of Oct4 (Octamer Binding Transcription Factor-4), which is another un-differentiated cell marker, were also evaluated through immunostaining with antibody against Oct4. The results are shown in FIG. 2.

The cells cultured in the non-conditioned medium (NCM) showed differentiated shapes (b in FIG. 2A). However, the cells cultured in the NCM containing Gö6983 (PKC inhibitor) were not differentiated and maintained typical hESC colony shapes (c in FIG. 2A). And also, the cells cultured in the NCM containing Gö6983 showed significant increases in both alkaline phosphatase and Oct4 expression levels (f in FIG. 2A and c in FIG. 2B). hESCs cultured in conditioned medium (CM) was shown here as a control for undifferentiated hESC colonies (a, d in FIG. 2A and a, d in FIG. 2B).

### EXAMPLE 2

### Evaluation of Differentiation of Stem Cells in the Presence of PKC Activator

(1) Culture in mTeSR-1 Medium Containing PKC Activator

Colony clumps (5 colony clumps) of human embryonic stem cells (CHA6 cell line, CHA Medical University, Korea) were seeded to each wells of a 96-well plate coated with Matrigel (BD Biosciences, USA). A undifferentiation-maintaining medium, i.e., mTeSR-1 medium containing bFGF, TGF-beta, LiCl, GABA, and pipecolic acid (StemCell Technologies, USA), was added to the each wells, which were then cultured at about 37° C., in about 5.0% CO₂ incubator. The culture was performed for 5 to 7 days, while replacing the medium with a new medium containing 10 nM of 12-O-tetradecanoylphorbol-13-acetate (TPA) as a PKC activator, everyday. As a control, human embryonic stem cells were cultured in the mTeSR-1 medium without the PKC activator. And also, human embryonic stem cells were cultured in the mTeSR-1 medium treated with both 10 nM of TPA and 5 uM of Go6983.

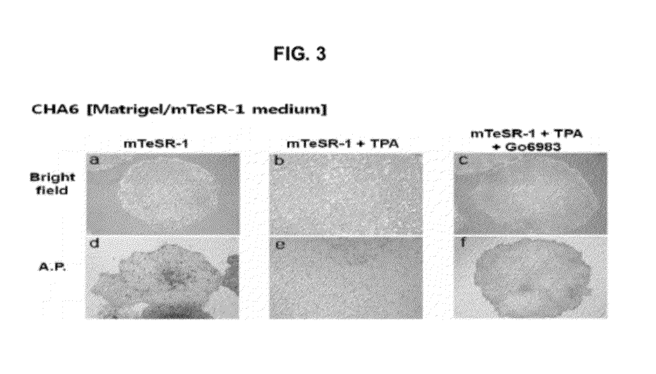

For evaluating differentiation of the stem cells, existence and distribution of the cells were shape of the cells was observed with an optical microscope (a to c in FIG. 3). The cells cultured in the mTeSR-1 medium formed colony shapes, which were conglomerating each other, without differentiation (a in FIG. 3). In contrast, the cells cultured in the mTeSR-1 medium containing the PKC activator (i.e., TPA) were differentiated and broadly distributed (b in FIG. 3).

As a result of alkaline phosphatase staining, the staining level and colony shape of the cells cultured in the mTeSR-1 medium were similar to those of undifferentiated stem cells (d in FIG. 3). However, the cells cultured in the mTeSR-1 medium containing the PKC activator (TPA) showed very low alkaline phosphatase staining level and were broadly distributed (e in FIG. 3).

And also, the cells cultured in the mTeSR-1 medium treated with both TPA and Gö6983 showed shapes similar to undifferentiated stem cells (c and f in FIG. 3). It is thought that these results were due to inhibition of the differentiation by the PKC inhibitor (Gö6983).

(2) Culture in Other Mediums Containing PKC Activator

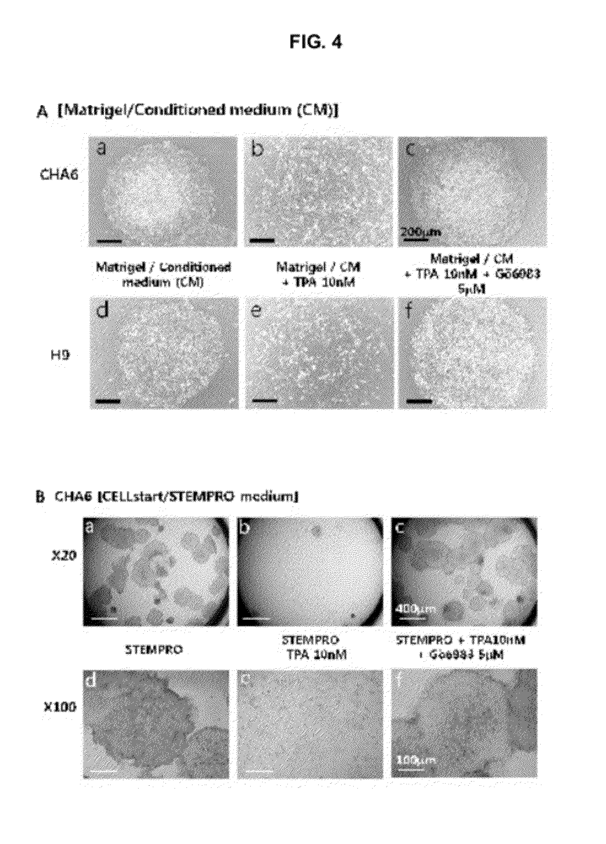

Evaluations of differentiation were also performed in accordance with the same procedures as in the above (1), using other undifferentiation-maintaining mediums, [i.e., Matrigel (extracellular matrix)/MEF-conditioned medium (Home made; Serum-free media [80% (v/v) DMEM/F12 (GIBCO), 20% (v/v) Knockout serum replacement (GIBCO), 1% (v/v) MEM non-essential amino acids (GIBCO), 0.1 mM β-mercaptoethanol (GIBCO), 1% (v/v) Pennicillin/streptomycin (Caisson)] was conditioned by γ-irradiated CF-1 mouse embryonic fibroblasts (MEF) at 37° C. for 24 hours) and CELLstart (extracellular matrix) (Invitrogen)/and STEMPRO (Invitrogen)], instead of the mTeSR-1 medium. In addition, differentiation of other hESC, i.e., H9 hESC line (WiCell, Wis., USA) was also evaluated in the Matrigel (extracellular matrix)/MEF-conditioned medium with a PKC activator. The results are shown in FIG. 4.

The PKC activator also induced differentiation of hESCs, even when other undifferentiation-maintaining mediums were used (b and e in FIG. 4A; and b and e in FIG. 4B). The induction of differentiation by the PKC activator was also observed in other hESCs, i.e., H9 hESC line (b and e in FIG. 4A).

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A medium for maintaining stemness of stem cells comprising a protein kinase C inhibitor in a basal medium.

2. The medium of claim 1, wherein the protein kinase C inhibitor is selected from the group consisting of 2-[1-(3-dimethylaminopropyl)-5-methoxyindol-3-yl]-3-(1H-indol-3-yl)maleimide; 3-[1 -[3-(dimethylamino)propyl]-5-methoxy-1H-indol-3-yl]-4-(1H-indol-3-yl)-1H-p yrrole-2,5-dione; 3-(1H-indol-3-yl)-4-[2-(4-methylpiperazin-1-yl)quinazolin-4-yl]pyrrole-2,5-dione); 3-{1[3-(amidinothio)propyl]-1H-indol-3-yl}-3-(1-methyl-1H-indol-3-yl)maleimide methane sulfonate; 13-hydroxyoctadecadienoic acid; bisindolylmaleimide; 2-[1-(3-dimethylaminopropyl)indol-3-yl]-3-(indol-3-yl) maleimide; 2,6-diamino-N-([1-(1-oxotridecyl)-2-piperidinyl]methyl)hexanamide; and 4′-demethylamino-4′-hydroxystaurosporine.

3. The medium of claim 1, wherein the protein kinase C inhibitor is contained in an amount of 0.001 to 1000 uM.

4. The medium of claim 1, wherein the basal medium is selected from the group consisting of DMEM (Dulbecco's Modified Eagle's Medium), MEM (Minimal Essential Medium), BME (Basal Medium Eagle), RPMI 1640, DMEM/F-12 (Dulbecco's Modified Eagle's Medium: Nutrient Mixture F-12), DMEM/F-10 (Dulbecco's Modified Eagle's Medium: Nutrient Mixture F-10), α-MEM (α-Minimal essential Medium), G-MEM (Glasgow's Minimal Essential Medium), IMDM (Isocove's Modified Dulbecco's Medium), and KnockOut DMEM.

5. The medium of claim 1, wherein the basal medium is a xenopathogen-free medium.

6. The medium of claim 1, wherein the stem cells are embryonic stem cells or adult stem cells.

7. The medium of claim 1, wherein the stem cells are selected from the group consisting of neural stem cells, hepatic stem cells, hematopoietic stem cells, umbilical cord blood stem cells, epidermal stem cells, gastrointestinal stem cells, endothelial stem cells, muscle stem cells, mesenchymal stem cells, and pancreatic stem cells.

8. A medium for inducing differentiation of stem cells comprising a protein kinase C activator in a basal medium.

9. The medium of claim 8, wherein the protein kinase C activator is selected from the group consisting of 12-O-tetradecanoylphorbol-13-acetate; 1-hexylindolactam-V10; 6,11,12,14-tetrahydroxy-abieta-5,8,11,13-tetraene-7-one; 8-octyl-benzolactam-V9; acetyl-1-carnitine; phorbol 12-myristate 13-acetate; (R)-7-cyano-2,3,4,5-tetrahydro-1-(1H-imidazol-4-ylmethyl)-3-(phenylmethyl)-4-(2-thienylsulfonyl)-1H-1,4-benzodiazepine; Bryostatin 1; 1-oleoyl-2-acetyl-sn-glycerol; L-a-phosphatidylinositol-3,4-bisphosphate; 1-stearoyl-2-arachidonoyl-sn-glycerol; and resiniferonol 9,13,14-ortho-phenylacetate.

10. The medium of claim 8, wherein the protein kinase C activator is contained in an amount of 0.001 to 1000 uM.

11. The medium of claim 8, wherein the basal medium is selected from the group consisting of DMEM (Dulbecco's Modified Eagle's Medium), MEM (Minimal Essential Medium), BME (Basal Medium Eagle), RPMI 1640, DMEM/F-12 (Dulbecco's Modified Eagle's Medium: Nutrient Mixture F-12), DMEM/F-10 (Dulbecco's Modified Eagle's Medium: Nutrient Mixture F-10), α-MEM (α-Minimal essential Medium), G-MEM (Glasgow's Minimal Essential Medium), IMDM (Isocove's Modified Dulbecco's Medium), and KnockOut DMEM.

12. The medium of claim 8, wherein the basal medium is a xenogen-free medium.

13. The medium of claim 8, wherein the stem cells are embryonic stem cells or adult stem cells.

14. The medium of claim 8, wherein the stem cells are selected from the group consisting of neural stem cells, hepatic stem cells, hematopoietic stem cells, umbilical cord blood stem cells, epidermal stem cells, gastrointestinal stem cells, endothelial stem cells, muscle stem cells, mesenchymal stem cells, and pancreatic stem cells.

15. A method for culturing stem cells while maintaining stemness thereof, which comprises culturing the stem cells in the medium according to claim 1.

16. The method of claim 15, wherein the culturing the stem cells is performed under the feeder-free condition.

17. A method for inducing differentiation of stem cells, which comprises culturing the stem cells in the medium according to claim 8.

18. The method of claim 17, wherein the culturing the stem cells is performed under the feeder-free condition.
